(19) United States
(12) Patent Application Publication
(10) Pub. No.: US 2011/0177615 A1
(43) Pub. Date: Jul. 21, 2011

(54) PROTEIN EXPRESSION

(76) Inventor: Neal Anthony Eric Hopkins, Wiltshire (GB)

(21) Appl. No.: 13/059,705

(22) PCT Filed: Aug. 19, 2009
(86) PCT No.: PCT/GB09/02025

§ 371 Date: Mar. 28, 2011

(30) Foreign Application Priority Data
Aug. 19, 2008 (GB) 0815039.3

(51) Int. Cl. G01N 33/68; C12P 21/02; C07K 19/00; C07H 21/00; C12N 15/63; C07K 17/00

(52) U.S. Cl. 436/501; 435/68.1; 530/350; 536/23.4; 435/320.1; 530/402

(57) ABSTRACT

The present invention is concerned with improved expression of recombinant proteins, in particular fusion proteins, and methods for their expression. The present invention is also concerned with proteins for use in detection assays, and especially for directed surface immobilisation.

The present invention is concerned with expression of recombinant proteins, in particular fusion proteins, and methods for their expression. The present invention is also concerned with proteins for use in detection assays, and especially for directed immobilisation to surfaces.

The expression and production of recombinant proteins, especially correctly folded recombinant proteins, requires processes in which a number of parameters are required to be finely tuned, such as choice of host and expression vector, cultivation temperature, and considering use of a co-factor, chaperone or fusion partner. One particular difficulty encountered in expression of recombinant proteins is that of obtaining correct and efficient folding of recombinant proteins in the expression host, and consequently production of good yields of correctly folded protein. Often, the protein should be correctly folded within the expression host such that a soluble functionally active protein is expressed, produced and isolated.

Microorganisms such as Escherichia coli are outstanding hosts for recombinant expression of proteins, with transfection of such a host commonly through an expression vector or plasmid encoding for the desired protein. Other expression hosts include yeasts such as Pichia pastoris, and mammalian cells such as CHO cells. The expression may result in a high yield of a correctly-folded, soluble protein, however more often than not the yield will be low and/or the protein will be incorrectly folded. Moreover, the recombinant protein may form insoluble aggregates known as inclusion bodies, which are generally misfolded and thus biologically inactive. The nature, rate and level of expression of a protein can influence the formation of inclusion bodies. This is believed to be a consequence of insufficient time, or absence of folding chaperones, for a nascent protein to fold into the correctly folded conformation. Proteins that contain strongly hydrophobic or highly charged regions are more likely to form inclusion bodies (Mukhopadhyay, Adv. Biochem. Eng. Biotechnol., 1997, 56, 61-109). Though proteins formed in inclusion bodies can be folded in-vitro subsequent to isolation and purification this folding may be neither optimum nor provide the correctly folded conformation, and thus expression and correct folding in-vivo is both favoured and desired.

Expression of aggregation prone proteins often requires the use of a molecular chaperone which can interact reversibly with nascent polypeptide chains during the protein folding process to prevent aggregation. Alternatively, co-expressing a fusion partner with the desired protein may improve the quality of expression. A fusion partner is a peptide or polypeptide of molecular weight normally less than 30 kDa in size expressed covalently linked to the desired protein, usually at the C-terminus or N-terminus of the protein. The product of expressing a protein covalently linked to a fusion partner is known as a fusion protein. Expressing a fusion partner with a protein can provide advantages such as improved protein yield, prevented proteolysis or increased protein solubility. The fusion partner should however be carefully chosen to provide the desired effect. For example, highly charged fusion partners may result in the protein being expressed in inclusion bodies. Such a fusion partner should therefore be avoided if a high yield of correctly folded soluble protein is required.

A fusion partner may also add functionality to the protein to aid in purification, or to enable immobilisation of the protein to surfaces, such as the surface of a biosensor. Such a fusion partner may also be described as an affinity tag, which is a chemical or biological molecule which enables purification and/or attachment to a surface via a functional chemical group. Fusion partners often also have the ability to be cleaved from the recombinant protein via suitable proteases. Among the most potent fusion partners are the E. coli maltose binding protein (MBP) and the E. coli N-utilizing substance A (NusA). MBP and NusA act as solubility enhancing partners and are especially suited for the expression of proteins prone to form inclusion bodies.

The use of proteins as recognition elements, for example in ELISA assays or on biosensor surfaces, is well known and has wide application in the fields of biochemistry and microbiology. Such proteins include antibodies, antibody fragments, lectins, and enzymes. Recognition elements can be immobilised to surfaces via functional chemical groups situated on the surface of the recognition element, or alternatively via an affinity tag covalently linked to the recognition element and presenting such functional groups. Examples of functional groups suitable for surface immobilisation include primary and secondary amine groups, carboxyl groups, and thiol groups. An affinity tag may contain molecules such as biotin, which can bind to streptavidin coated surfaces.

Surface immobilisation of proteins via a thiol functional group often necessitates use of a thiol-containing affinity tag. The reason for this is that thiol functional groups in proteins are usually unavailable for use in immobilisation as they are often situated towards the core of the protein, and thus are sterically unavailable, and more often than not are bound to another thiol group in the protein via a disulphide bond. Consequently, immobilisation via thiol functional groups is usually through use of an affinity tag comprising a thiol functional group. As used herein, directed immobilisation is immobilisation of a recognition element via either a single specific chemical functional group, or alternatively a single specific affinity tag. The affinity tag may for example be a peptide. Immobilisation via thiol functional groups thus has less inherent problems than immobilisation via either amine or carboxyl groups. Amine and carboxyl groups are abundantly present at the surface of a protein with the result that immobilisation is not at any specific functional group, but is random or non-directed. Furthermore, in such a random or directed approach the biological activity of the protein may be modified, diminished or even lost. However, though use of affinity tags having thiol groups for directed immobilisation is one of the favoured approaches for immobilisation of proteins, the linking of such affinity tags to proteins can be difficult, both chemically (synthetically) and biologically. In particular, expression of such an affinity-tagged recombinant protein often produces a low yield of protein and/or the formation of inclusion bodies. For example, a single chain antibody expressed to comprise a biotin mimic peptide, having amino acid sequence PCHPQFPRCYAL, is expressed in inclusion bodies (Das et al, J. Virol. Methods, 2004, 117, 169-177), and Schmiedl et al (J. Immunol. Methods, 2000, 242, 101-114) have found that replacing a free C-terminal cysteine by serine in an expressed fusion antibody provides a 3 to 5 fold increase in protein yield, and a significant increase in the biological activity of the antibody produced.

There is thus a requirement for alternative and improved methods for expression of recombinant proteins, especially affinity-tagged recombinant proteins wherein the affinity tag has a thiol functional group, and particularly to produce a good yield of correctly-folded recombinant protein.

The present application is thus directed to improving the expression yield of recombinant proteins, and in particular recombinant proteins suitable for directed surface immobilisation via thiol functional groups.

Accordingly, in a first aspect, the present invention provides a method for expressing a fusion protein comprising transfecting an expression host with a nucleic acid encoding for the fusion protein, wherein the fusion protein comprises a fusion partner, and the fusion partner comprises two cysteine residues and an amino acid sequence corresponding to that of a peptide substrate for a disulphide isomerase in the expression host.

In a preferred embodiment, the amino acid sequence corresponding to that of the peptide substrate comprises the two cysteine residues.

As used herein, and throughout the art, a fusion protein is the product of coexpressing a protein with a fusion partner. The fusion partner is covalently linked to the protein, either directly or indirectly through a linking chemical molecule. An equivalent product to a fusion protein may also be produced through chemical synthesis. A fusion antibody is the product of coexpressing an antibody with a fusion partner.

The Applicant has found that an amino acid sequence corresponding to that of a peptide substrate for a disulphide isomerase can be used as, or as part of, a fusion partner in the expression of recombinant proteins. Such a fusion partner provides for good expression of correctly folded protein. It is believed that such a fusion partner, in association with the host disulphide isomerase, enables the protein to be guided along the correct folding pathway, with the result of producing high yields of correctly folded protein. The peptide substrate moiety of the fusion partner is also believed to function as a solubility enhancing partner during expression, providing increased yields of soluble protein. Examples, of disulphide isomerase substrates include the hexapeptide CYIQNC (Gilbert et al, Biochemistry, 1989, 28, 7298), the 28 residue peptide FCLEPPYTGPSKARYFYYNAKAGLCQ (Darby et al, Biochemistry 1994, 33, 7937), and somatostatin with sequence AGCKNFFWKTFTSC (Klappa et al, Eur. J. Biochem. 1998, 273, 24992).

In a preferred embodiment, the protein moiety of the fusion protein comprises at least one cysteine residue, more preferably at least two cysteine residues, and most preferably at least two cysteine residues which form a disulphide bond in the correctly folded protein. Though the solubility and yield of proteins devoid of cysteine residues may be improved by expressing the protein with a fusion partner comprising an amino acid sequence of a disulphide isomerase in the expression host, the fusion partner function is particularly directed to aiding folding through oxidation and reduction of disulphide bonds.

Native disulphide bond formation is a complex process whereby disulphide bonds are formed (oxidation) and incorrect bonds are either broken (reduction) or rearranged (isomerisation). The enzymes chiefly involved with this process in both eukaryotes and prokaryotes are disulphide oxidoreductases/isomerases. Protein disulphide isomerase (PDI) is a disulphide isomerase found in eukaryotic cells and DsbA and DsbC (periplasmic protein thiol:disulphide oxidoreductases) disulphide isomerases found in prokaryotic cells. PDI is found in all eukaryotic systems studied to date, and DsbA has been found in many Gram-negative bacteria, including E. coli and Vibrio cholerae. Disulphide isomerases have also been used as chaperones in cell-free expression systems, whereby addition of the disulphide isomerase was shown to increase the solubility by 50% (Merck et al, J. Biochem., 1999, 125, 328-333). The active site (catalytic domain) for both these enzymes comprises two cysteine residues in the sequence C—X—Y—C, wherein the two amino acids X and Y play a major role in determining the function of the enzyme. A peptide substrate for a disulphide isomerase is a peptide which can bind to the active site of the enzyme such that the enzyme can catalyse thiol-disulphide exchange reactions in the peptide.

A preferred feature of a peptide substrate for a disulphide isomerase is two cysteine residues capable of forming an unstrained disulphide bond, and preferably wherein the two cysteine residues are linked by a disulphide bond. The two cysteine residues may be separated by intervening amino acid residues, such as 1, 2, 3, 4, 5, 6, 7, 8, 9, 10, 11, 12, 13, 14, 15, 16, 17, 18, 19, 20, 30, 40 or 50 intervening amino acid residues, though in a preferred embodiment the cysteine residues are separated by at least four amino acid residues, for example the sequence SQGS is a known sequence that separates cysteine residues in a disulphide isomerase substrate. The peptide substrate preferably comprises at least one tryptophan or tyrosine residue within its amino acid sequence. Any acidic amino acid (i.e. aspartic acid or glutamic acid) in the amino acid sequence of the disulphide substrate is preferably at least one non-acidic amino acid from any tryptophan or tyrosine residue. A peptide substrate preferably comprises a maximum of 100 amino acids, more preferably a maximum of 30 amino acids, and most preferably a maximum of 10 amino acids. A peptide substrate may comprise no or minimal secondary structure. One particular peptide substrate specific for PDI, DsbA and DsbC has the amino acid sequence NRCSQGSCWN (Ruddock L. W. et al, Biochem J. 1996, 315, 1001 to 1005). The Applicant has found that utilisation of a fusion partner comprising this peptide substrate in the expression of recombinant proteins may result in yields of correctly folded protein higher than that achieved without a fusion partner. Further amino acid sequences for substrates to disulphide isomerases are to be found in the art. Additional sequences may also be identified and/or designed by using techniques known in the art. For example, additional peptide substrates could be identified by phage displayed affinity selection from diverse peptide libraries, and could be targeted to a particular disulphide isomerase. Amino acid sequences for substrates for disulphide isomerases include those peptides having amino acid sequences comprising single or multiple changes in amino acid residues, or deletions of amino acid residues, or insertions of additional amino acid residues from/within a known disulphide isomerase peptide substrate sequence, subject to the sequence retaining its ability to undergo thiol-disulphide exchange reactions within the active site of a disulphide isomerase.

In a preferred embodiment, the method is directed to the expression of a soluble fusion protein as soluble fusion proteins will not require solubilisation, and in general will be correctly folded, and thus not require in-vitro folding.

A fusion partner that comprises a substrate for a disulphide isomerase in the expression host, which substrate also comprises two cysteine residues capable of forming an unstrained disulphide bond (i.e. two thiol functional groups) has particularly been shown to provide good yields of recombinant protein capable of being immobilised to a surface via a thiol functional group, and especially through directed immobilisation. Moreover, the yields are significantly higher than that for fusion partners comprising single thiol functional groups, or fusion partners comprising multiple thiol functional groups but which are devoid of an amino acid sequence of a peptide substrate for a disulphide isomerase. Until now expression of affinity-tagged proteins wherein the affinity tag comprises a thiol group has resulted in particularly low yields of the protein. The method of the first aspect overcomes this deficiency in the art.

The expression host may be any known expression host, however in a preferred embodiment the expression host is a strain of E. coli or a strain of yeast, and in a more preferred embodiment is either E. coli BL21 (DE3) or the yeast Pichia pastoris. The Applicant has in particular shown that coexpression of antibodies with a fusion partner in either E. coli BL21 (DE3) or the yeast Pichia pastoris, wherein the fusion partner comprises a peptide substrate for a disulphide isomerase in the particular expression host, results in a good yield of correctly folded antibody.

The nucleic acid encoding for the fusion protein may be delivered to the expression host in a suitable plasmid or expression vector, and thus in one embodiment of the method the expression host may be transfected with a plasmid or expression vector comprising the nucleic acid encoding for the fusion protein.

In a second aspect, the present invention provides a protein having a covalently linked partner, wherein the partner is a peptide comprising two cysteine residues an amino acid sequence corresponding to that of a peptide substrate for a disulphide isomerase.

The partner is preferably covalently linked to either the C-terminus or N-terminus of the protein. The protein may be suitable for use in a detection assay. The partner may be capable of functioning as an affinity tag. A partner having at least one thiol functional group can for example function as an affinity tag and be used for directed immobilisation to surfaces. Such a partner having at least one thiol functional group may also enable labelling of the protein such as with a fluorescent group. The labelled protein could be used for example in an identification assay, or in tracing movement of the protein within a eukaryote or a prokaryote.

In a preferred embodiment the protein having a covalently linked partner is a fusion protein, wherein the partner is a fusion partner, and the fusion protein is obtainable through expression in an expression host having the disulphide isomerase. The fusion protein is more preferably expressed by the method of the first aspect.

A partner, or fusion partner, comprising two cysteine residues linked through an unrestrained disulphide bond may be immobilised to a surface containing thiol functional groups by first reducing the disulphide bond and then linking/binding one of the subsequent free thiol groups to the thiol functional group on the surface. Alternatively, the disulphide bond may be cleaved by illuminating the protein with ultra violet light such as described in International Patent Application PCT/DK2004/000047. The mechanism for this process relies on an electron transfer mechanism between aromatic amino acid residues, such as tryptophan, and the disulphide linkage.

In a third aspect, the present invention provides a nucleic acid encoding for a fusion protein, wherein the fusion protein comprises a fusion partner and the fusion partner comprises an amino acid sequence of a peptide substrate for a disulphide isomerase, and in a fourth aspect, the present invention provides an expression vector or plasmid encoding for expression of the fusion protein, wherein the expression vector or plasmid comprises the nucleic acid of the third aspect.

The nucleic acid may also encode for a chemical linking group in the fusion protein situated between the fusion partner and the protein. A chemical linking group may be required if the fusion protein is to be used for directed surface immobilisation wherein availability to a functional group in the fusion partner would otherwise be sterically hindered. Alternatively, a chemical linking group may contribute to minimising non-specific binding of the protein to the surface, through distancing the protein from the surface.

The fusion partner, or where applicable the chemical linking group, may be expressed such that it is covalently linked to either the C-terminus or N-terminus of the protein.

If the protein is an antibody intended for directed surface immobilisation then the fusion partner, or chemical linking group, is usually expressed linked to the C-terminus of the antibody as the binding domains of antibodies immobilised at their N-terminus are often not optimally presented for binding a target species.

The present invention will now be described with reference to the following non-limiting examples and drawings in which

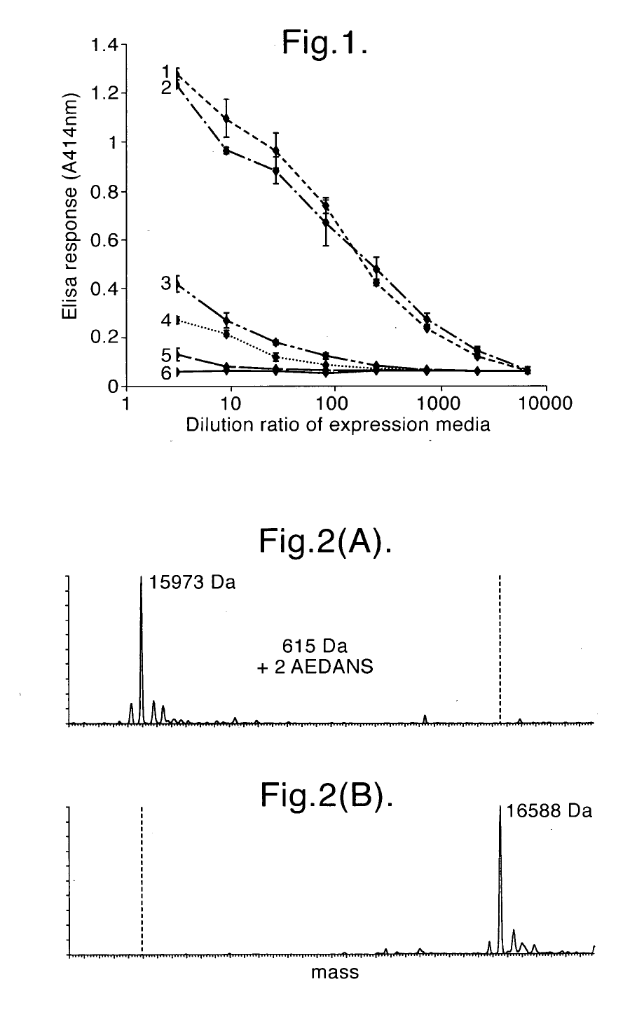

FIG. 1 is a graph representing the binding of a selection of recombinant ovalbumin fusion antibodies to ovalbumin in an ELISA assay. The x-axis represents the dilution factor of expression media containing the recombinant fusion antibodies, and the y-axis the response in ELISA with absorbance measured at 414 nm;

FIGS. 2a and b are mass spectra for a fusion protein both before (FIG. 2a), and after (FIG. 2b) reduction and fluorescent labelling with IAEDANS;

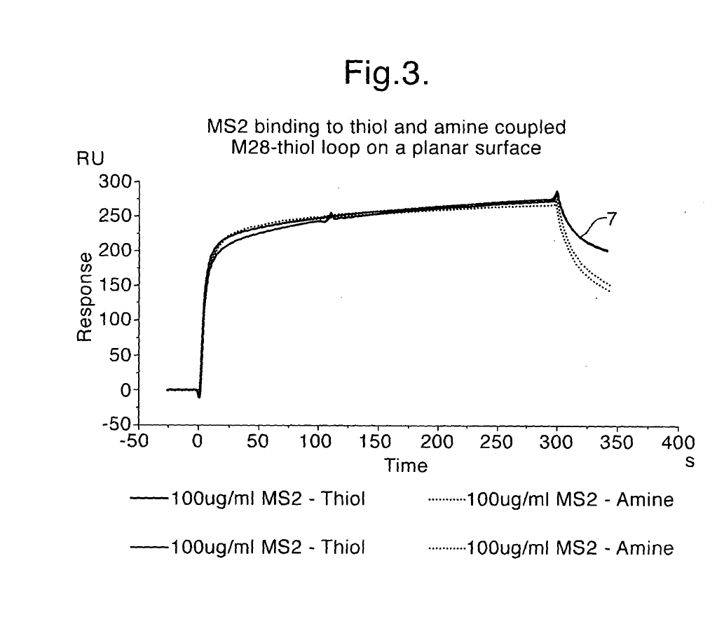

FIG. 3 is a graph representing the binding (association and dissociation) of MS2 to fusion antibodies which have been immobilised to a BIAcore® sensor surface by either amine coupling or thiol coupling.

### EXAMPLES

Fusion protein expression and purification used a culture media of 2×yeast/tryptone (YT) liquid media containing 0.1% glucose and 30 μg/ml chloramphenicol, an expression media of 2×YT liquid media containing 0.1% glucose, 30 μg/ml chloramphenicol and 1 mM IPTG (isopropyl-beta-D-thiogalactopyranoside), a periplamic extraction buffer of 50 mM Tris-HCl buffer (pH 7.5) and 1 mM EDTA containing 20% (w/v) sucrose, and ÄKTA™ chromatography buffer A of 40 mM Tris-HCl and 750 mM NaCl (pH 8.0), and buffer B of 40 mM Tris-HCl and 750 mM NaCl (pH 8.0) containing 500 mM imidazole.

Escherichia coli BL21 was cultured in culture media at 37° C., shaking at 200 rpm, for 18 h. The cultures were centrifuged, resuspended in culture medium and an optical density of 50 at 600 nm achieved by adding 50% glycerol (v/v). The final suspension was used to inoculate expression media, using appropriate volumes of expression media to achieve a starting optical density (600 nm) of 0.05. The inoculated culture was incubated at 37° C. and shaken at 180 rpm until an optical density (600 nm) of more than 1.0 was reached, whereupon 1 mM IPTG was added, and the culture shaken at 180 rpm, 26° C. for 18 h. The cells were harvested by centrifugation at 10000 g for 30 min at 4° C., the supernatant was retained, and the cells were resuspended in ice-cold periplasmic extraction buffer to 5% of the initial culture volume, and incubated for 1 h with occasional shaking. The suspension was centrifuged at 13,000 rpm for 10 min at 4° C. and the supernatant (periplasmic extract) again retained. Both the culture supernatant and periplasmic extract were subsequently pooled and filter sterilised using a 0.2 μm filter, followed by buffer exchange into phosphate buffered saline, and concentrated to a final volume of approximately 50 ml. The fusion proteins comprised a terminal histidine tag to aid in purification, which used a 1 ml His-Trap Nickel column on an ÄKTA™ chromatography system. Samples from the chromatography were analysed using ELISA (i.e. soluble periplasmic extract and clarified media) and purified by dialysis in phosphate buffered saline at 5° C. For the ELISA assay ovalbumin in phosphate buffered saline (10 μg/ml; 100 μl) was used to coat ELISA plates overnight at 4° C. and then washed extensively. Each fusion protein preparation (50 μl) was added to phosphate buffered saline containing 0.05% Tween 20 (100 ul) in the first well of the plate, and then triple diluted in each subsequent well.

Expression of recombinant fusion proteins in Pichia pastoris used the EasySelect™ Pichia Expression Kit (Invitrogen), the pPICZ plasmid and Zeocin™. The methods were as detailed in the manual of methods for expression of recombinant proteins (Invitrogen, Catalog No. K1740-01).

### Example 1

Referring now to FIG. 1 the expression and purification of recombinant ovalbumin fusion antibodies in Escherichia coli BL21, wherein each antibody comprised a different fusion partner was evaluated using ELISA assays specific for ovalbumin. A high binding response is representative of a high yield of correctly folded antibody. The plotted data is an average from two experiments. Curve 1 was obtained for a fusion antibody having a fusion partner comprising the amino acid sequence NRCSQGSCWN (SEQ ID 1), a known substrate of a disulphide isomerase in E. coli. The fusion antibody also comprised a ‘hinge’ chemical linking group of amino acid sequence GGSGGAP. The fusion partner further comprised amino acid sequences AS and LQ either side of the peptide of SEQ ID 1. A histidine tag HHHHHH was provided to enable purification of the fusion antibody. The fusion antibody has the amino acid sequence as shown in SEQ ID 2. Curve 2 was obtained for a fusion antibody consisting solely of a histidine tag as fusion partner, and no chemical linking group. Curve 3 corresponds to a fusion antibody having SEQ ID 3 and wherein the fusion partner comprises a biotin acceptor peptide having a biotin attached. Curve 4 corresponds to a fusion antibody having SEQ ID 4 wherein the fusion partner comprises a biotin mimic peptide. The biotin mimic peptide, with amino acid sequence PCHPQFPRCYAL, is an example of a peptide comprising two cysteine residues but which is not a known substrate for a disulphide isomerase. Curve 5 corresponds to a fusion antibody having SEQ ID 5 wherein the fusion partner comprises a single cysteine residue. Curve 6 was the control with no antibody present.

Until now expression of antibodies suitable for controlled/directed immobilisation to surfaces has proven difficult, with yields often very low. In particular antibodies with an affinity tag comprising a single thiol functional group have been problematic, especially in terms of low yield and/or misfolding of the protein, as illustrated by the data of FIG. 1. The yield of such proteins has been significantly lower than that for the native antibody, or the antibody labelled only with a histidine tag. However, an affinity tag/fusion partner comprising SEQ ID 1, which is the amino acid sequence of a known substrate for a disulphide isomerase found in a diverse range of organisms including E. coli, enables a good yield of correctly folded labelled antibody to be produced. The yield of correctly folded fusion antibody is at least equal, if not slightly elevated, to that of the fusion antibody having a fusion partner consisting solely of a histidine tag.

Affinity tags/fusion partners having an amino acid sequence comprising two cysteine residues but which is not a substrate for a disulphide isomerase, such as the biotin mimic peptide, do not provide good yields (i.e. similar yields to the native protein or protein labelled with only a histidine tag) of correctly folded protein. Such yields are often less than 50% of that for the native protein or protein consisting solely of a histidine tag as fusion partner. The yield of the antibody having a fusion partner comprising the biotin mimic peptide was only 20% of that for the antibody consisting solely of a histidine tag as fusion partner, possibly due to production of inclusion bodies.

### Example 2

A similar experiment to that with E. coli BL21 (example 1) but with expression of the fusion antibody of SEQ ID 6, comprising the 5A7 IgNARv antibody, in the yeast Pichia pastoris resulted in an even more surprising result with a yield of 1.330 mg fusion protein from 250 ml of growth medium, as compared to a yield of only 0.245 mg from 250 ml of growth medium for the fusion antibody consisting solely of a histidine tag as fusion partner. The yield was therefore more than 5-fold higher.

### Example 3

Having regard to FIG. 2, the fusion antibody having SEQ ID 6 was reduced with 10 mM Tris(2-carboxyethyl)phosphine.HCl (TCEP) for 10 mins and then labelled with 10 mM N-(iodoacetyl)-N′-(5-sulfo-1-napthyl)ethylenediamine (IAEDANS, a fluorescent dye). The resulting product was analysed by electrospray ionisation mass spectrometry. FIG. 2a is a mass spectrum of the fusion antibody before reduction and labelling, and FIG. 2b the labelled fusion antibody. The data shows that two molecules of AEDANS (MW 307.5) had been added to the antibody, and that labelling of the two cysteine residues was 100% since no non-labelled, or single labelled fusion protein was evident from the mass spectrum. Moreover, the fluorescently-labelled antibody retained its full binding activity, thus indicating that none of the six cysteine residues within the antibody had been disrupted or labelled, and that only the two cysteine residues within the fusion partner were available for labelling, and thereby for attachment to a sensor surface.

### Example 4

Having regard to FIG. 3, the fusion antibody having SEQ ID 7, comprising a single chain antibody specific to the virus stimulant MS2, was immobilised to a BIAcore® sensor surface via (i) amine coupling, and (ii) thiol coupling, and the interaction with MS2 monitored. Even though, the density of protein immobilised by amine coupling was 2.25 times that immobilised by thiol coupling both surfaces produced similar levels of response, and the thiol coupled surface 7 better levels of activity as apparent from the association and dissociation rates of the binding curves shown. In light of the labelling and mass spectrometry result of Example 3 the Applicant believes that only the cysteine residues of the fusion partner are available for attachment to the surface, and thus directed coupling of the antibody via thiol coupling has been achieved. The amine coupling is random, and thus not directed.

Sequence Listing

SEQ ID NO: 1
NRCSQGSCWN
SEQ ID NO: 2
DYKDIVMTQSPASLAVSLGQRATISCRASQSVSTSSYSYMHWYQQKPGQ
PPKLLIKYASNLESGVPARFSGSGSGTDFTLNIHPVVEEDTATYYCQHS
WEIPYTFGGGTKLEIKRGGGGSGGGGSEVKLMESGGGLVKPGGSLKLSC
AASGFTFSSYAMSWVRQTPEKRLEWVATIRSGGSYTYYPDSVKGRFTIS
RDNAKNTLYLQMSSLRSEDTAMYYCARHRMITTAGDAMDYWGQGTSVTV
SAASGAGGSGGAPASNRCSQGSCWNLQHHHHHH
SEQ ID NO: 3
DYKDIVMTQSPASLAVSLGQRATISCRASQSVSTSSYSYMHWYQQKPGQ
PPKLLIKYASNLESGVPARFSGSGSGTDFTLNIHPVVEEDTATYYCQHS
WEIPYTFGGGTKLEIKRGGGGSGGGGSEVKLMESGGGLVKPGGSLKLSC
AASGFTFSSYAMSWVRQTPEKRLEWVATIRSGGSYTYYPDSVKGRFTIS
RDNAKNTLYLQMSSLRSEDTAMYYCARHRMITTAGDAMDYWGQGTSVTV
SAASGAGGSGGAPASGGGLNDIFEAQKIEWHELQHHHHHH
SEQ ID NO: 4
DYKDIVMTQSPASLAVSLGQRATISCRASQSVSTSSYSYMHWYQQKPGQ
PPKLLIKYASNLESGVPARFSGSGSGTDFTLNIHPVVEEDTATYYCQHS
WEIPYTFGGGTKLEIKRGGGGSGGGGSEVKLMESGGGLVKPGGSLKLSC
AASGFTFSSYAMSWVRQTPEKRLEWVATIRSGGSYTYYPDSVKGRFTIS
RDNAKNTLYLQMSSLRSEDTAMYYCARHRMITTAGDAMDYWGQGTSVTV
SAASGAGGSGGAPASPCHPQFPRCYALQHHHHHH
SEQ ID NO: 5
DYKDIVMTQSPASLAVSLGQRATISCRASQSVSTSSYSYMHWYQQKPGQ
PPKLLIKYASNLESGVPARFSGSGSGTDFTLNIHPVVEEDTATYYCQHS
WEIPYTFGGGTKLEIKRGGGGSGGGGSEVKLMESGGGLVKPGGSLKLSC
AASGFTFSSYAMSWVRQTPEKRLEWVATIRSGGSYTYYPDSVKGRFTIS
RDNAKNTLYLQMSSLRSEDTAMYYCARHRMITTAGDAMDYWGQGTSVTV
SAASGAHHHHHHC
SEQ ID NO: 6
ARVDQTPRSVTKETGESLTINCVLRDASYALGSTCWYRKKSGEGNEESI
SKGGRYVETVNSGSKSFSLRINDLTVEDGGTYRCGLGVAGGYCDYALCS
SRYAECGDGTAVTVNAASGAGGSGGAPASNRCSQGSCWNLQHHHHHH
SEQ ID NO: 7
MADYKDIVLTQSPAIMSASPGEKVTMTCSASSSVSYMHWYQQKSGTSPK
RWIYDTSKLASGVPARFSGSGSGTSYSLTISSMEAEDAATYYCHQRSSY
PWTFGGGTKLELKRGGGGSGGGGSGGGGSGSGGSEVKLMESGGDLVKPG
GSLKLSCAASGFIFRSYGMSWVRQTPDKRLEWVATTSSGGSYTYYPDSV
KGRFTISRDNAKNTLYLQMSSLKSEDTAMYYCARRGYDDNYAMDYWGQG
TSVTVSSASGAGGSGGAPASNRCSQGSCWNLQHHHHHH
SEQ ID NO: 8
CYIQNC
SEQ ID NO: 9
FCLEPPYTGPSKARYFYYNAKAGLCQ
SEQ ID NO: 10
AGCKNFFWKTFTSC

1. A method for expressing a fusion protein, comprising transfecting an expression host with a nucleic acid encoding the fusion protein, wherein the fusion protein comprises a fusion partner, and the fusion partner comprises two cysteine residues and an amino acid sequence of a peptide substrate for a disulphide isomerase in the expression host.

2. A method of claim 1, wherein the expression host is transfected with an expression vector comprising the nucleic acid encoding the fusion protein.

3. A method of claim 1, wherein the fusion protein is a soluble fusion protein.

4. A method of claim 1, wherein the amino acid sequence of the peptide substrate comprises the two cysteine residues.

5. A method of claim 4, wherein the two cysteine residues are separated by at least four amino acid residues.

6. A method of claim 5, wherein the two cysteine residues are separated by amino acid sequence SQGS.

7. A method of claim 1, wherein the fusion partner comprises amino acid sequence NRCSQGSCWN (SEQ ID NO:1).

8. A method of claim 1, wherein the expression host is a strain of Escherichia coli.

9. A method of claim 1, wherein the expression host is the yeast Pichia pastoris.

10. A method of claim 1, wherein the fusion protein comprises an antibody.

11. A fusion protein comprising a fusion partner, wherein the fusion partner comprises two cysteine residues and an amino acid sequence of a peptide substrate for a disulphide isomerase.

12. The fusion protein of claim 11, wherein the fusion partner is covalently linked to either the C-terminus or N-terminus of the protein.

13. The fusion protein of claim 11, wherein the amino acid sequence of the peptide substrate comprises the two cysteine residues.

14. A fusion protein of claim 13, wherein the two cysteine residues are separated by at least four amino acid residues.

15. A fusion protein of claim 13, wherein the two cysteine residues are separated by amino acid sequence SQGS.

16. A fusion protein of claim 1, wherein the fusion partner comprises amino acid sequence NRCSQGSCWN (SEQ ID NO:1).

17. The fusion protein, wherein the fusion protein is obtainable by the method of claim 1.

18. A nucleic acid encoding the fusion protein of claim 11.

19. An expression vector, wherein the expression vector comprises the nucleic acid of claim 18.

20. A method of using the fusion protein of claim 11, comprising providing the fusion protein in a detection assay.

21. A method of directed surface immobilization, comprising immobilizing the fusion protein of claim 11 via a thiol functional group.

22. A method for expressing a protein, comprising providing a peptide substrate of a disulphide isomerase in an expression host comprising the disulphide isomerase.
